(19) United States
(12) Patent Application Publication
(10) Pub. No.: US 2013/0098361 A1
(43) Pub. Date: Apr. 25, 2013

(54) Method and Device for Supplying at Least One Medical Gas to a Patient Receiving Artificial Respiration with the Aid of a Ventilator

(71) Applicants: Rainer Köbrich, Neulussheim (DE); Thilo Joost, Friedberg (DE)

(72) Inventors: Rainer Köbrich, Neulussheim (DE); Thilo Joost, Friedberg (DE)

(73) Assignee: MAQUET VERTRIEB UND SERVICE DEUTSCHLAND GMBH, Rastatt (DE)

(21) Appl. No.: 13/643,851

(22) PCT Filed: Nov. 30, 2010
(86) PCT No.: PCT/EP10/68558

§ 371 Date: Jan. 7, 2013

(30) Foreign Application Priority Data
Apr. 29, 2010 (DE) 10 2010 016 699.5

(51) Int. Cl. A61M 16/12; A61M 16/10; A61M 16/00

(52) U.S. Cl. CPC A61M 16/12; A61M 16/0057; A61M 16/104
USPC 128/203.14

(57) ABSTRACT

A device and method for administering at least one medical gas (NO) to a patient mechanically ventilated by means of a ventilator. The ventilator produces a respiratory gas flow (O₂/N₂) in a feed line. Gas pulses of the medical gas (NO) are supplied to said respiratory gas flow. The gas pulses are produced and fed into a patient feed line by means of at least two regulating means arranged in parallel. Firstly, the temporal start of at least one future inhalation phase of the patient is determined. Subsequently, the regulating means are controlled by means of a control unit such that a gas pulse is fed into the patient feed line at the determined start of the future inhalation phase. To achieve that the therapy gas is fed into without delay in case of very short inhalation times either nitrogen (N₂) or helium (He) is used as carrier gas for the active substance (NO).

### CROSS REFERENCE TO RELATED APPLICATIONS

This application is entitled to the benefit of and incorporates by reference essential subject matter disclosed in International Patent Application No. PCT/EP2010/068558 filed on Nov. 30, 2010 and German Patent Application No. 10 2010 016 699.5 filed Apr. 29, 2010.

### FIELD OF THE INVENTION

The invention relates to a method and a device for administering at least one medical gas to a mechanically ventilated patient. A machine ventilator produces a respiratory gas flow in a first line supplying respiratory gas. A predetermined amount of a medical gas to be administered is added to the respiratory gas flow. At least the gas exhaled by the patient and the proportion of the respiratory gas introduced into the first line by the ventilator and the medical gas fed into a patient feed line which have not been inhaled by the patient are discharged via a second line.

### BACKGROUND OF THE INVENTION

Documents EP 0 937 479 B1, EP 0 937 479 B1, U.S. Pat. No. 5,558,083, EP 0 786 264 B1, EP 1 516 639 B1, and EP 0 723 466 B1 disclose devices and methods for delivering nitrogen monoxide in a continuous and pulsed manner over the course of time to a mechanically ventilated patient. Control valves for setting the amount of nitrogen monoxide are provided, which valves, however, as a function of the design in each case, allow a defined amount of gas to pass through per unit time under specific, defined pressure conditions. Therefore, there is reliance on providing the medical gas in a combination appropriate for the device and on providing the patient with an amount of gas appropriate for the treatment in the opened state of the regulating means. However, it is desirable to wean the mechanically ventilated patient, if necessary, from the active ingredients of the medical gas in a continuous or stepwise manner and to reduce the amount of the administered medical gas per unit time. In the case of high, gas source-provided concentrations of the medical gas and in the case of a very low amount of gas to be supplied, a precise metering of low amounts of gas is therefore necessary, whereas in the case of gas sources having a low concentration of the medical gas and administration of relatively large amounts of the medical gas by means of the regulating means, substantially larger amounts of gas have to be introduced into the first line.

### SUMMARY OF THE INVENTION

It is an object of the invention to specify a method and a device for administering at least one medical gas to a patient mechanically ventilated by means of a ventilator, in which method and device the amount of gas to be administered is easily adjustable.

This object is achieved by a method having the features of claim 1 and by a device having the features of the independent device claim. Advantageous developments of the invention are specified in the dependent claims.

What is achieved by the method and the device for administering at least one medical gas to a patient mechanically ventilated by means of a ventilator is that, via each of the two regulating means arranged in parallel, the medical gas can be introduced into the ventilator-produced respiratory gas flow in the first line and can thus be supplied to the patient. By opening one of the regulating means or both regulating means to produce the gas pulses, it is possible to set the amount, more particularly the volume, of the medical gas introduced into the patient feed line with an appropriate selection of the pulse length and pulse repetition. Firstly, the temporal start of at least one inhalation phase of the patient is determined. Subsequently, the regulating means are controlled by means of a control unit such that a gas pulse is fed into the patient feed line at the determined start of the inhalation phase.

Feeding at the start of the inhalation phase can be understood to mean feeding precisely at the temporal start of the inhalation phase or at a preset temporal interval after the start of the inhalation phase. Here, the temporal interval is particularly selected such that the gas pulse is fed during the inhalation phase, preferably within the first third of the inhalation phase.

Preferably, only this one gas pulse is fed during the inhalation phase. More particularly, during the apnea phase following the inhalation phase and the exhalation phase following it, no medical gas is fed. What is achieved by this is that the entire gas to be taken up by the patient during the inhalation phase is already supplied to the respiratory gas at the start of the inhalation phase and can be taken up by the patient. This means that, firstly, no medical gas is wasted during the apnea phase and/or the exhalation phase and, secondly, the medical gas can thus reach deep into the lungs and thereby be taken up more effectively.

The medical gas is supplied especially in a burst at the start of the inhalation phase, so that as much medical gas as possible can be taken up by the patient from the start of the inhalation. Supplying in a burst is especially understood to mean that the amount of medical gas to be supplied is supplied within a very short time with a large volume flow.

By appropriately selecting the dimensions of the regulating means, gas sources containing different concentrations of the medical gas can thus also be used, without requiring structural modifications of the device for administering the medical gas. Thus, both the method and the device provide variable adjustment of the amount of gas to be administered in large adjustment ranges and thus a broad concentration spectrum. The pulse-shaped partial pressure brought about by the gas pulses is measurable into the airways of the mechanically ventilated patient.

It is advantageous when the regulating means are controlled by means of a or the control unit such that an amount of gas defined in relation to a gas pulse and/or gas volume defined in relation to a gas pulse is fed into the patient feed line. As a result, the amount of gas or the gas volume that is required for the administration can be introduced into the patient feed line in a simple manner.

It is particularly advantageous when the medical gas contains NO (nitrogen monoxide). The medical gas can in particular be provided as a gas mixture composed of NO (nitrogen monoxide) and N₂ (nitrogen). A gas mixture composed of NO (nitrogen monoxide) and He (helium) has also been found to be particularly advantageous, since especially helium can achieve particularly short reaction and response times. As a result, effective administration is possible especially in the case of newborn babies and in the case of premature babies and the relatively low amounts of the mixture composed of respiratory gas and medical gas that are inhaled by these patients.

It is further advantageous to have more than two regulating means arranged in parallel. Experiments have shown that it is particularly advantageous to have four regulating means arranged in parallel, wherein the regulating means are formed such that at least two of the regulating means in the opened state allow a different amount of gas to pass through. The regulating means arranged in parallel are preferably valves and are then also referred to as a valve bank.

In this connection, it has been found to be particularly advantageous when, under the defined pressure conditions, the first valve has a flow of 0.16 liters per minute, valve 2 has a flow of 1.6 liters per minute and valves 3 and 4 each have a flow of 8 liters per minute when opened constantly (measured using medical air). It is further advantageous when regulating means are used which have a shortest realizable opening time of 7 milliseconds, preferably in the range from 4 milliseconds to 7 milliseconds. The control unit can open the valves individually or in any desired combination, and so, in the case of the specific exemplary embodiments, a maximum flow of 17.76 liters per minute is possible.

It is particularly advantageous when the control unit optimizes the opening of the regulating means to the effect that a very short opening time is achieved within one breath. As a result, a targeted injection of the medical gas into an initial portion of the breath is achieved. Also achieved as a result is a large adjustable metering range of the medical gas to be administered to the patient.

In one embodiment, 18.60 microliters of the medical gas are administered when only one valve having a flow of 0.16 liters per minute, 7 milliseconds opening time per gas pulse per breath theoretically, is opened. However, owing to the required valve stroke and/or the response delay, 13 microliters are administered in practical experiments using these parameters. Even in the case of a premature baby, which has a tidal volume of 2 ml, it is possible as a result to set a low concentration of 0.1 ppm with a starting concentration of the medical gas of 1000 ppm. As a result, after a more highly concentrated administration of the medical gas, it can be reduced in a stepwise or continuous manner to approximately 676 microliters per minute, and weaning of the patient from the medical gas or from the active ingredient thereof is therefore easily possible. Furthermore, the use of multiple regulating means connected in parallel makes it possible, at the administered concentrations, i.e., target concentrations, which are currently conventional, to also use more highly concentrated supply gas sources, with the result that said supply gas sources, more particularly supply gas cylinders, have to be exchanged at greater intervals, and as a result logistics and consumption costs can be lowered. Alternatively or additionally, the invention makes a larger therapeutic concentration spectrum clinically available.

As already mentioned, it is advantageous when the regulating means in an opened state allow volume flows differing face to face to pass through from the gas source to the patient feed line. In the case of more than two regulating means, it is advantageous when at least two of the regulating means in the opened state allow different volume flows to pass through from the gas source to the patient feed line. As a result, a concentration from a relatively large concentration spectrum can be set in a simple manner.

It is particularly advantageous when the regulating means each comprise at least one solenoid valve. Furthermore, a restricting orifice or another restricting means for limiting the volume flow flowing through the regulating means can be arranged upstream and/or downstream of at least one regulating means. Solenoid valves are, firstly, inexpensive and, secondly, solenoid valves have relatively short response times. The solenoid valves are controlled in particular in a binary manner, and so they are completely closed in a first operating state and completely opened in a second operating state. By means of the restricting means for limiting the volume flow flowing through the regulating means, it is possible to use regulating means of the same type, more particularly solenoid valves of the same type, wherein the volume flow flowing through the regulating means in the opened state differs owing to the provision of different flow resistances. As a result, it is easily possible to produce different volume flows through the regulating means.

In an advantageous development of the invention, gas is removed from the second line. At least the proportion of the medical gas and/or the proportion of a reaction product of the medical gas in the removed gas is determined. The gas can be removed from the second line via a measurement line and supplied to an analysis unit for the detection of at least the proportion of the medical gas and/or the proportion of a reaction product of the medical gas. More particularly, the removal and detection can be carried out once or more than once during one act of inhalation, preferably repeatedly during each act of inhalation. As a result, the concentration of the medical gas in the exhaled gas can be easily determined, monitored and/or regulated. The inner diameter of the measurement line is preferably smaller than the diameter of the first line, the second line and the patient feed line.

It is further advantageous to compare the determined proportion of the medical gas, as the actual value, with a target value and, in the event of a determined deviation of the actual value from the preset target value, to adapt the amount of the medical gas introduced into the patient feed line during the gas pulse as a function of the comparative result. Preferably, the proportion of the medical gas in the exhaled gas is regulated to the preset target value. As a result, the amount of the medical gas to be administered to the patient can be easily monitored, and/or kept constant. If, in addition to or as an alternative to the proportion of the medical gas, the proportion of a reaction product of the medical gas is analyzed, it is advantageous to determine the proportion of an oxidation product of the medical gas. If nitrogen monoxide (NO) is used as medical gas, the proportion of the oxidation product nitrogen dioxide (NO₂) can be determined in particular. The proportion of the determined nitrogen dioxide can then be compared with a permissible target value. When the target value is exceeded, the feeding of the medical gas into the patient feed line can then be stopped or the volume of the fed medical gas can be reduced. In the event of an excessively high concentration of nitrogen dioxide in the mechanical ventilation gas, the patient can be harmed, and so this must be avoided.

It is further advantageous when the ventilator determines information concerning a flow profile of the respiration of the mechanically ventilated patient. The control unit determines the start of the inhalation phase as a function of said flow profile or determines the time of the start of a future inhalation phase, preferably the next one. What is achieved by determining the start of the future inhalation phase from the flow profile determined by means of the ventilator is that it is not necessary to use a separate sensor for determining the start of the future inhalation phase. More particularly, it is not necessary to use a separate additional sensor for determining pressure conditions within the connecting piece and/or within the patient feed line. Thus, a simple inexpensive assembly is achieved.

In a preferred embodiment of the invention, the temporal start of multiple inhalation phases is determined. Exactly one gas pulse is fed at each start of each inhalation phase. Thus, what is achieved is that, at the start of each inhalation phase, the amount of medical gas required for the uptake of the amount of medical gas to be administered is provided, more particularly in a burst. Thus, the patient can take up, with each breath, the amount of medical gas that is optimal for his or her therapy.

Preferably in addition to the inhalation phase, at least one exhalation phase and/or at least one apnea phase are determined from the flow profile. A gas pulse containing medical gas is not fed into the patient feed line either during the exhalation phase or during the apnea phase. Thus, wastage of the medical gas is avoided, since the medical gas cannot be taken up by the patient either during the apnea phase or during the exhalation phase and it would thus be discharged unused via the second line.

In a preferred embodiment of the invention, data containing information concerning the times of at least two preceding inhalation phases are determined. The temporal start of the future inhalation phase is determined as a function of said data. In a particularly preferred embodiment of the invention, the start of the future inhalation phase is defined on the basis of more than two preceding inhalation phases. To determine the start of the inhalation phase, in particular the time interval between the start of the last two inhalation phases is determined and the start of the future inhalation phase is determined by adding said interval to the time of the start of the last inhalation phase.

In an alternative embodiment, the start of the inhalation phase can also be determined from the gradient of the flow profile. In this case, the start of a future inhalation phase is not determined, but rather the start of the current inhalation phase.

It is further advantageous when the gas volume of at least one preceding inhalation phase is determined. The control unit determines, as a function of said gas volume, the gas volume of the inhalation phase, and defines the amount of medical gas which is fed via the gas pulse at the start of the inhalation phase as a function of the determined gas volume. It is advantageous when the gas volume of multiple preceding inhalation phases is determined and the gas volume of a future inhalation phase and thus also the amount of medical gas to be fed during the inhalation phase are defined as a function of the gas volumes of the multiple preceding inhalation phases. To determine the gas volume of the future inhalation phase, it is, for example, possible to use pattern recognition in preceding respiratory phases. Alternatively, the gas volume of the future inhalation phase can be determined by averaging the preceding gas volumes of the preceding inhalation phases.

The amount of medical gas to be fed is defined in particular in proportion to the determined gas volume of the inhalation phase. The gas volume of the preceding inhalation phase or the gas volumes of the preceding inhalation phases are determined in particular from the flow profile determined by means of the ventilator. Thus, it is not necessary to provide an additional separate gas sensor for determining the gas volume.

The solenoid valves used are preferably valves switchable between a completely closed and a completely opened position, which valves are controlled in a binary manner.

The invention can be used especially in neonatology for treating pulmonary hypertension of a premature baby with nitrogen monoxide. Nitrogen monoxide is also administered in order to treat patients after organ transplantations. However, the invention can also be used for administering other gaseous medicaments.

Depending on the clinical use, up to 10% of the inspired volume can originate from a gas source for providing gaseous medicaments. Such a gas source is also referred to as an additive gas source, since it is provided in addition to a respiratory gas source or oxygen source. The invention avoids the disadvantage in the prior art that a delay time arises from the time of measuring the flow velocity of the respiratory gas used for the inspiration of the patient up to the mechanical adjustment of a control valve used for feeding the medical gas, and that there is an occurrence of relatively large concentration fluctuations of the administered medical gas in the mechanically ventilated air provided to the patient with dynamic flow profiles. Furthermore, in the case of known control valves, the control range of the conducted medical gas is limited relatively strongly. In the case of valves which allow a large flow of the medical gas, low flow rates can only be set relatively imprecisely. In a further embodiment of the invention, discontinuous feeding by means of multiple gas pulses into the respiratory air of the ventilator patient circuit comprising the first line, the second line and the patient feed line can be carried out.

Further features and advantages of the invention are found in the following description, which more particularly elucidates the invention by means of exemplary embodiments in conjunction with the attached figures.

The following is shown:

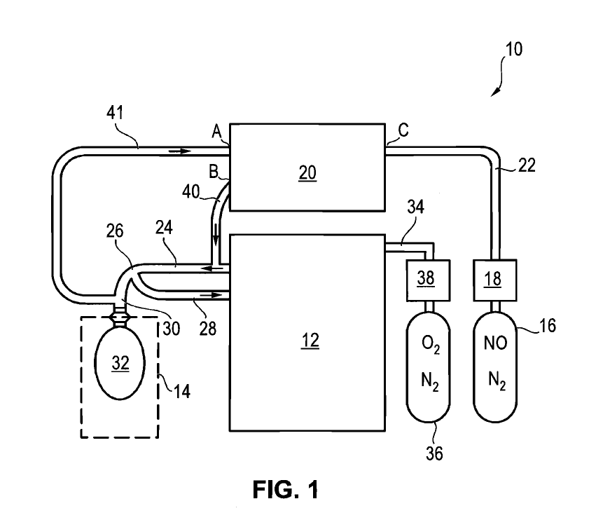

FIG. 1 a diagram of a device for administering at least one medical gas to a patient mechanically ventilated by means of a ventilator according to a first exemplary embodiment;

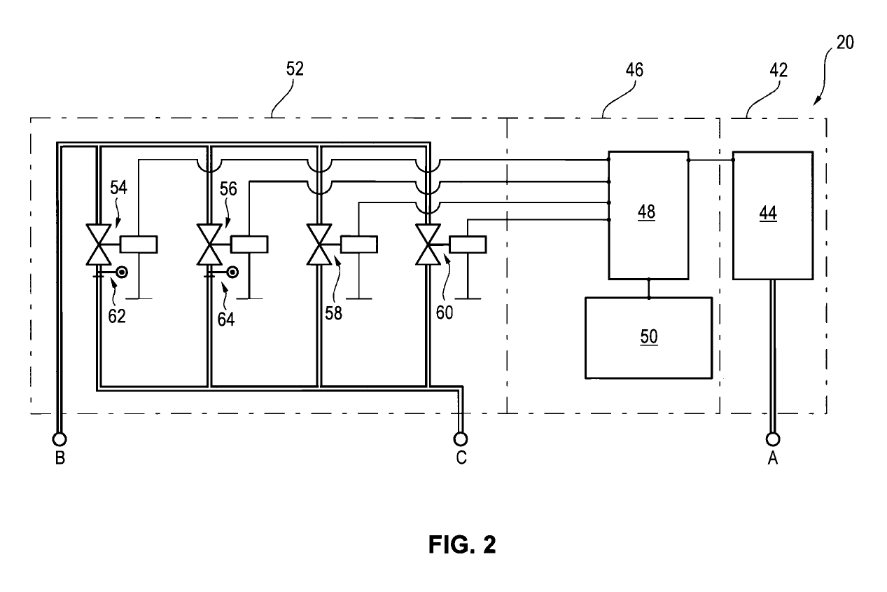

FIG. 2 a diagram of components of an administering apparatus for administering the medical gas;

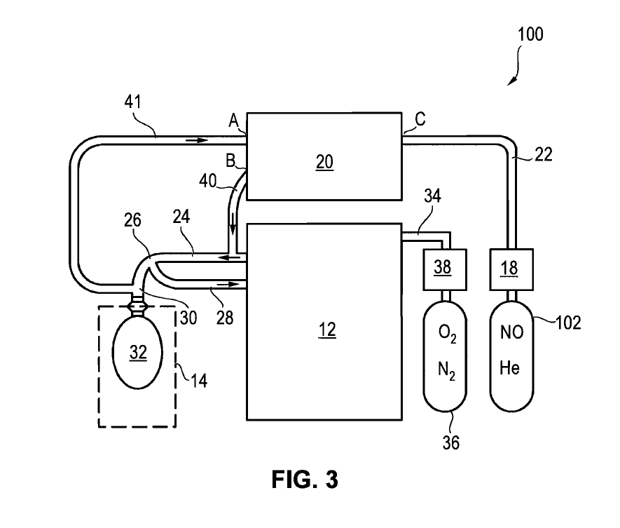

FIG. 3 a diagram of a device for administering at least one medical gas to a patient mechanically ventilated by means of a ventilator according to a second exemplary embodiment of the invention;

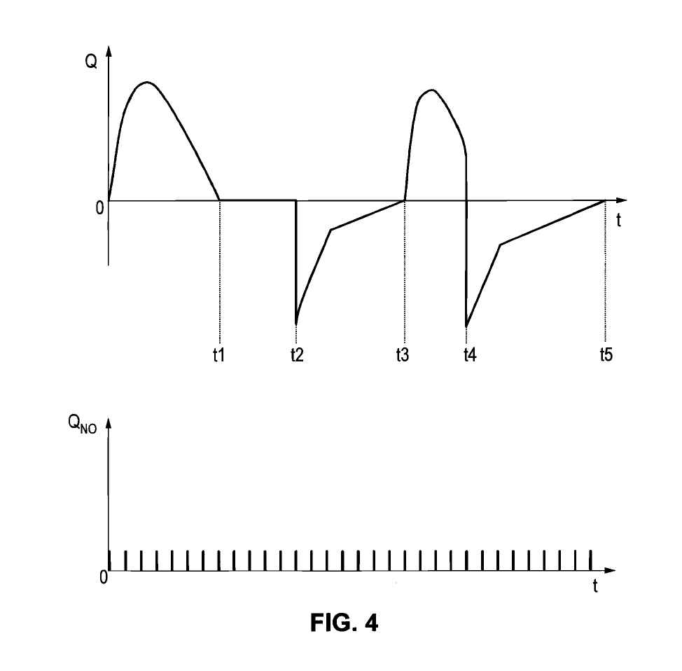

FIG. 4 a representation of the temporal course of the respiration of the mechanically ventilated patient and of the administration of the medical gas according to the first and second exemplary embodiment of the invention;

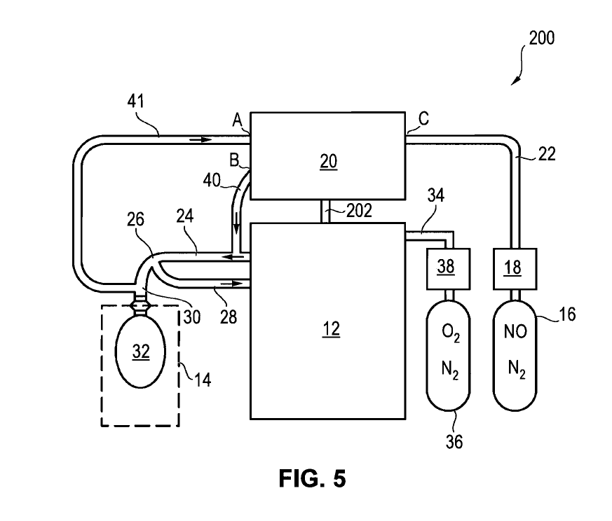

FIG. 5 a diagram of a device for administering at least one medical gas to a patient mechanically ventilated by means of a ventilator according to a third exemplary embodiment of the invention;

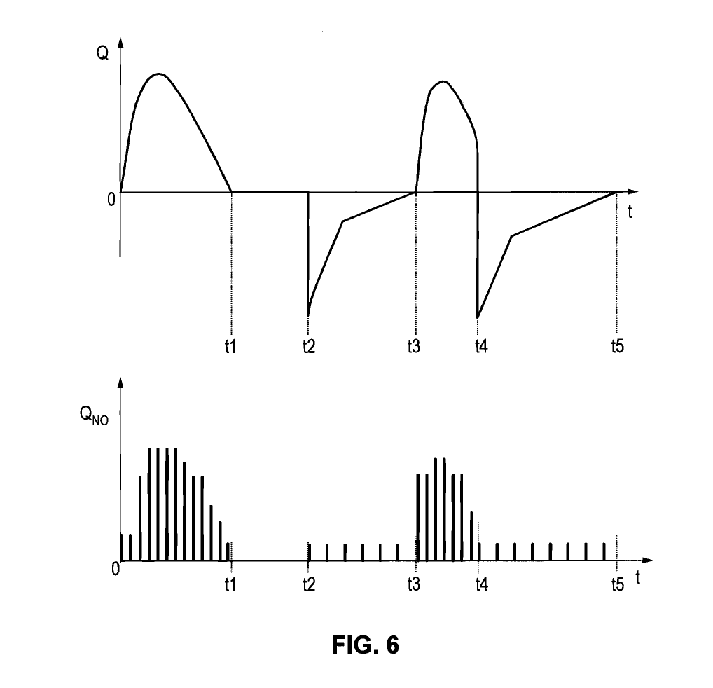

FIG. 6 a representation of the temporal course of the respiration of a mechanically ventilated patient and of the administration of the medical gas according to the third exemplary embodiment of the invention;

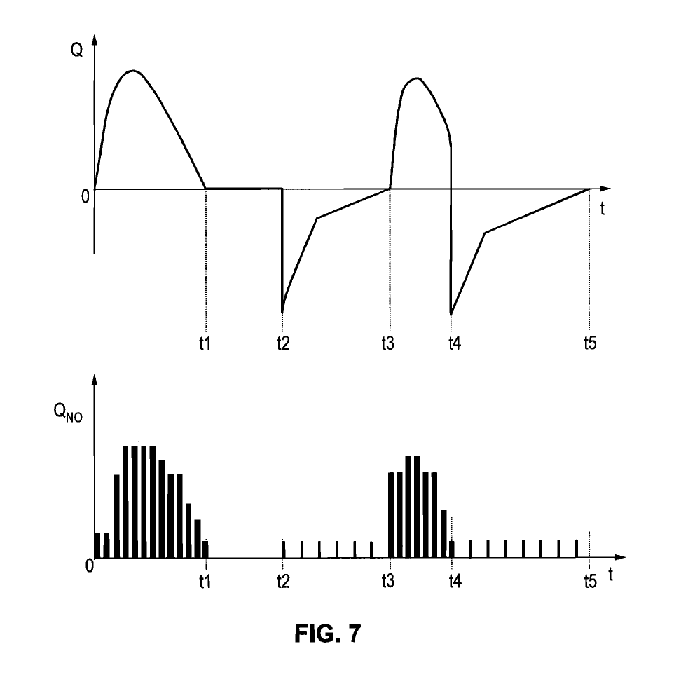

FIG. 7 a representation of the temporal course of the respiration of a mechanically ventilated patient and of the administration of the medical gas according to a fourth exemplary embodiment of the invention;

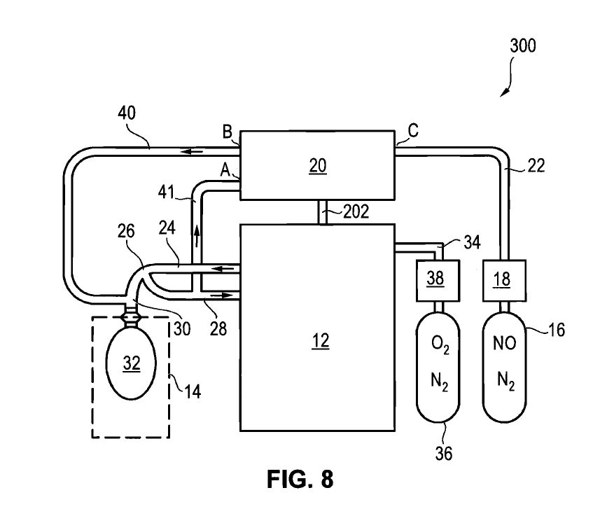

FIG. 8 a diagram of a device for administering at least one medical gas to a patient mechanically ventilated by means of a ventilator according to a fifth exemplary embodiment of the invention;

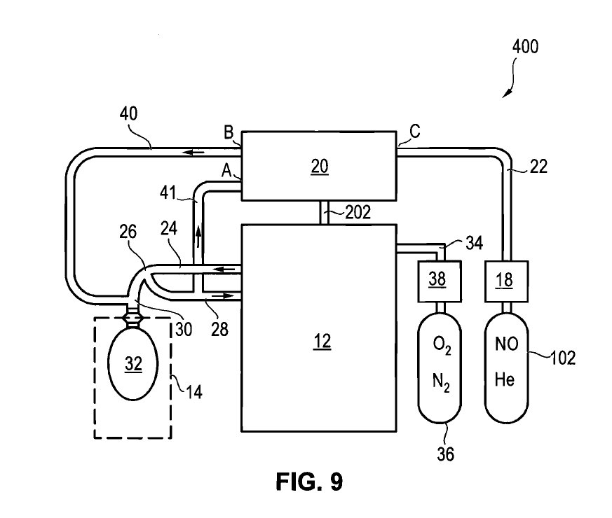

FIG. 9 a diagram of a device for administering at least one medical gas to a patient mechanically ventilated by means of a ventilator according to a sixth exemplary embodiment of the invention; and

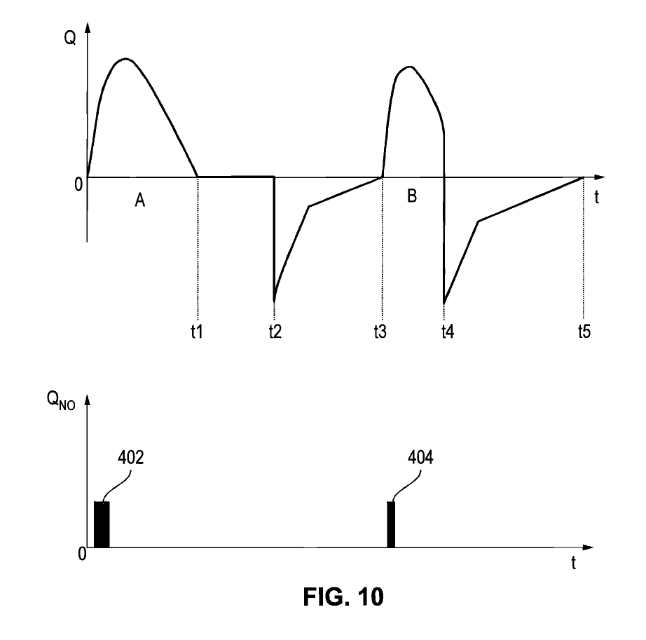

FIG. 10 a representation of the temporal course of the respiration of a mechanically ventilated patient and of the administration of the medical gas by means of the device according to FIG. 8 or FIG. 9.

### DETAILED DESCRIPTION OF THE PREFERRED EMBODIMENTS

FIG. 1 shows a diagram of a device 10 for administering at least one medical gas to a patient 14 mechanically ventilated by means of a ventilator 12 according to a first exemplary embodiment of the invention. In this exemplary embodiment, the medical gas used is NO (nitrogen monoxide). This gas is provided in a gas cylinder 16 as a gas mixture (NO/N₂) comprising N₂ nitrogen and NO nitrogen monoxide. By means of a pressure regulator 18, the gas mixture NO/N₂ is supplied to a metering device 20 via a connecting tube 22 having a target pressure, preset at the pressure regulator 18, at the connector C of the metering device 20. From the ventilator 12, a first line 24 designed as a respiratory air tube leads to a connecting element 26 designed as a Y-piece. In addition, a second line 28 designed as a waste air tube and a patient feed line 30 are connected to the connecting element 26. In the present exemplary embodiment, the patient feed line 30 is connected to a test lung, simulating the patient 14, in the form of an inflatable balloon 32. To mechanically ventilate a living patient 14, the end of the patient feed line 30 leading to the patient 14 is connected to a face mask or to a tube inserted into the airways of the patient 14. The waste air tube 28 is led back to the ventilator 12, wherein the gas mixture flowing back through the waste air tube 28 is either discharged or recycled in the ventilator 12. In the present exemplary embodiment, the ventilator 12 is connected to a gas source in the form of a gas cylinder 36 via a connecting tube 34. The gas cylinder 36 contains a gas mixture (O₂/N₂) comprising oxygen (O₂) and nitrogen (N₂). The gas mixture O₂/N₂ is limited to a preset target value by means of a pressure regulator 38 and supplied to the ventilator 12 via the connecting tube 34. In other exemplary embodiments, oxygen and nitrogen can also be provided by means of separate gas sources 36, more particularly also via a central gas supply in a hospital.

The ventilator 12 produces a constant flow of respiratory gas in the respiratory air tube 24. The medical gas mixture NO/N₂ determined for the treatment of the patient is supplied to this constant respiratory gas flow via the connecting line 40 by means of the metering device 20. For this purpose, the metering device 20 produces continuously gas pulses with a pulse frequency which is at least independent from the respiratory rate of the patient.

In addition, a measurement line 41 is connected to the patient feed line 30 and conducts at least some of the gas mixture situated in the patient feed line 30 to the connector A of the metering device 20. The gas mixture supplied to the metering device 20 via the connector A is analyzed by a measurement/evaluation unit 44 of the metering device 20.

FIG. 2 shows a diagram containing components of the metering device 20 according to FIG. 1. The metering device 20 is also referred to as an NO-administering apparatus because of the nitrogen monoxide used as medical gas in the exemplary embodiment. The metering device 20 has a first module containing a measurement/evaluation unit 44, which analyzes the proportion of NO in the gas mixture (O₂/N₂/NO) supplied via the connector A and transmits a corresponding measured value to a control unit 48 arranged in the second module 46. The control unit 48 is connected to an operating unit 50 in the form of a human-machine interface. The operating unit 50 is preferably designed as a touchscreen. Via the operating unit 50, it is possible to set parameters of the metering device 20, more particularly target values. In addition, set values, measured values and operating values can be output via a display unit of the operating unit 50. The control unit 48 is preferably connected to a control unit of the ventilator 12 via a data cable, which is not shown. Via this data cable, relevant parameters measured values and further information can be transmitted, preferably bidirectionally, between the control unit 48 and the control unit of the ventilator 12.

The metering device 20 has a third module 52, which, in the present exemplary embodiment, comprises four solenoid valves 54 to 60, which are each supplied with the medical gas mixture NO/NO₂ via the connector C. Upstream of the solenoid valves 54, 56 is, in each case, a metering orifice 62, 64 for restricting the flow through the respective solenoid valve 54, 56. The output sides of the solenoid valves 54 to 60 are connected to the connector B, and so the solenoid valves 54 to 60 are connected in parallel. The control unit 48 of the metering device 20 can individually control the solenoid valves 54 to 60, i.e., open them individually or in combination. Thus, it is possible to achieve a gas flow between the connector C and the connector B by opening a valve 54 to 60 and to thus feed medical gas NO via the connecting line 40 into the respiratory gas line 24. The amount of flow between the connector C and the connector B can be increased by the simultaneous opening of multiple valves 54 to 60. In addition, the administered amount, i.e., the amount of the medical gas NO fed into the respiratory gas line 24, can be set by appropriate selection of the pulse duration and/or by appropriate selection of the pulse frequency. In this connection, the gas pulses produced by the individual valves 54 to 60 can have a different pulse duration with preferably the same pulse frequency. The third module containing the parallel arrangement of multiple valves 54 to 60 is also referred to as valve bank 52. The valve bank 52 containing the four solenoid valves 54 to 60 allows a large adjustable metering range and flexible adaptation of the amount of gas to be administered when using gas sources 16 having different starting concentrations of the medical gas. The starting concentration is preferably preset as a parameter via the operating unit and taken into consideration when calculating the pulse duration and pulse frequency for producing the amount to be administered.

In the present exemplary embodiment, the solenoid valve 54 has a flow of 0.16 liters per minute, the solenoid valve 56 has a flow of 1.6 liters per minute and the solenoid valves 58 and 60 each have a flow of 8 liters per minute, measured using medical air. The pulse frequency, i.e. the clock rate, amounts to 104 gas pulses per minute, i.e. 104 bolups per minute. If smaller quantities of the medical gas NO shall be administered or for other reasons a lower clock rate and/or a lower pulse frequency shall be selected, said pulse frequency is preferably reduced to 52 gas pulses per minute or 26 gas pulses per minute. If a higher pulse frequency shall be selected, said pulse frequency can also be increased to 208 gas pulses per minute.

By means of the arrangement shown in FIG. 1, maximum starting doses of 40 ppm are administered in the case of adult patients and maximum starting doses of 20 ppm are administered in the case of children. In the case of newborn babies or premature babies, the maximum starting dose can be lower.

To wean the patient, the dose is lowered in a stepwise or continuous manner to 0.5 ppm; in the case of premature babies, to 0.1 ppm. The starting concentration of the medical gas in the gas source 26 is preferably 1000 ppm. All doses indicated refer to the respiratory air supplied to the Y-piece 26 and containing the introduced medical gas.

In general, the use of a valve bank 52 containing multiple valves 54 to 60 arranged in parallel makes it possible, in the case of currently conventional administered amounts, to use gas sources containing higher starting concentrations of the medical gas, more particularly up to 2000 ppm or up to 4000 ppm. Compared to gas sources containing 1000 ppm of the same amount of gas, the service lives are doubled when the starting concentration is doubled. Alternatively or additionally, the use of the valve bank 52 provides a larger therapeutic concentration spectrum. In the present exemplary embodiment, the minimum opening duration of the solenoid valves 54 to is 7 milliseconds. As a result, the amount of the medical gas NO fed into the respiratory gas line 24 can be varied in large ranges, resulting in a large adjustable therapeutic concentration spectrum.

FIG. 3 shows a diagram of a device 100 for administering at least one medical gas to a patient mechanically ventilated by means of a ventilator 12 according to a second exemplary embodiment of the invention. The device 100 matches the device 10 according to FIG. 1 in terms of structure and function. In contrast to FIG. 1, the medical gas nitrogen monoxide (NO) is provided as a gas mixture comprising nitrogen monoxide (NO) and helium (He). Preferably, the gas mixture (NO/He), apart from customary impurities, consists of nitrogen monoxide (NO) and helium (He). This gas mixture (NO/He) is provided by means of a gas source 102 in the form of a gas cylinder and supplied to the metering device 20 via the pressure regulator 18 and the connecting line 22 in the connector C. The gas mixture (NO/He) composed of nitrogen monoxide (NO) and helium (He) achieves very short response times. The gas pulses produced are immediately fed into the respiratory gas line 24.

It was found in experiments that the use of a gas mixture (NO/He) composed of nitrogen monoxide and helium, compared with the gas mixture (NO/N₂) used in the first exemplary embodiment according to FIG. 1 and composed of nitrogen monoxide and nitrogen, achieves a lower compression of the gas mixture (NO/He) composed of nitrogen monoxide and helium and thus achieves more direct feeding of the gas pulse into the respiratory air feed line. As a result, a corresponding pulse-like partial pressure increase is also measurable at the patient 14, and so in particular the pulse frequency of the gas pulses is perceptible by the patient 14. In the exemplary embodiment according to FIG. 1, a partial pressure increase brought about by the gas pulses is also measurable at the patient 14. However, in the case of identical gas pulses, the rise in the partial pressure at the patient 14 and the drop in the partial pressure after a gas pulse steeper when using the gas mixture NO/He than when using the gas mixture NO/N₂.

FIG. 4 shows representations of the temporal courses of the respiration of the mechanically ventilated patient 14 and the administration of the medical gas in the form of gas pulses. The upper graph shows the temporal course of the respiration of the patient 14 as volume flow Q. In the period between t0 and t1, a first inhalation phase of the patient 14 takes place. In the period between the times t1 and t2, apnea of the patient 14 occurs. Between the time t2 and t3, a first exhalation phase of the patient 14 takes place and, between the times t3 and t4, a second inhalation phase takes place which is shorter compared to the first inhalation phase. Between the times t4 and t5, a second exhalation phase takes place.

The second, lower graph shows the gas pulses fed into the respiratory air feed line 24 by means of the metering device 20 as volume flow of the relevant proportion of the medical gas NO. Supplying the medical gas in this exemplary embodiment is achieved by means of gas pulses having a constant pulse frequency and thus independently of the respiratory rate of the patient 14.

The solenoid valves used are preferably valves switchable between a completely closed and a completely opened position, which valves are controlled in a binary manner.

The invention can be used especially in neonatology for treating pulmonary hypertension of a premature baby with nitrogen monoxide. Nitrogen monoxide is also administered in order to treat patients after organ transplantations. However, the devices 10, 100 described in the exemplary embodiments can also be used for administering other gaseous medicaments.

It is further known to mix gaseous medicaments into a respiratory gas flow by means of a proportioning valve as a function of the flow velocity, measured in real-time by means of a flow meter, of the respiratory air flow.

FIG. 5 shows a diagram of a further device 200 for administering at least one medical gas to a patient 14 mechanically ventilated by means of a ventilator 12 according to a third exemplary embodiment of the invention. In contrast to the exemplary embodiments according to FIG. 1 and according to FIG. 3, the medical gas NO is metered into the patient circle part of the ventilator 12, i.e., into the respiratory air tube 24, in proportion to the respiratory course of the patient 14. In contrast to the exemplary embodiments according to FIGS. 1 and 3, in the third exemplary embodiment, gas pulses having different gas volumes are produced as a function of the respiratory phase and/or the course of the respiratory phase.

There is a data and/or signal cable 202 between the ventilator 12 and the metering device 20, via which information concerning a real-time flow profile of the respiration of the mechanically ventilated patient 14 transmits by means of signals and/or data to the control unit 48 of the metering device 20. For the data transmission, it is possible to use in particular a real-time-capable bus system, for example a CAN BUS or a serial interface, such as a USB interface or RS232 interface, using a real-time-capable data transmission protocol.

The medical gas is fed into the respiratory air feed line 24 such that, during the respiratory phases of the patient, a higher concentration of the medical gas is contained in the supplied ventilation air. In a first embodiment of the third exemplary embodiment, the gas pulses are delivered at a constant pulse frequency, wherein the amount of gas delivered per gas pulse is greater during the inhalation phases than during apnea phases and during the exhalation phases of the patient 14.

Alternatively or additionally, it is possible in further embodiments for the pulse frequency to be higher during the inhalation phases than during the exhalation phases and during apnea. In addition, it is possible during apnea of the patient 14 for the supplying of the medical gas by the metering device 20 to be interrupted. It is advantageous that by means of gas-pulse and pulse-frequency optimization performed by the control unit 44 or a control unit of the ventilator a relatively long opening time of the activated valves 54 to 60 is required within the defined pulse frequency. The pulse frequency is preferably 104 gas pulses per minute. Only when the required gas flow of the medical gas through the valve bank 52 is greater than or equal to the maximum flow through a valve 54 to 60, and so the flow through said valve 54 to 60 would not be sufficient to administer the required amount of the medical gas, or the valve 54 to 60 would no longer close and would thus produce no more gas pulses, is an additional further valve 54 to 60 or, instead of the first valve 54 to 60, a second valve 54 to 60 having a larger flow in the opened state controlled by the control unit 48.

In a fourth exemplary embodiment, in contrast to the exemplary embodiment shown in FIG. 5, the medical gas NO is not provided as a gas mixture composed of nitrogen monoxide and nitrogen (NO/N₂), but as a gas mixture composed of nitrogen monoxide (NO) and helium (He). The advantages associated with this gas mixture (NO, He) have already been elucidated in conjunction with FIG. 3. The gas pulses are produced in this fourth exemplary embodiment as described for the third exemplary embodiment in conjunction with FIG. 5.

FIG. 6 shows a representation of the temporal course of the respiration of the mechanically ventilated patient 14 and the temporal course of the administration of the medical gas (NO/N₂)/(NO/He). The upper graph shows the respiratory air flow of the mechanically ventilated patient 14, similar to FIG. 4, and the lower graph shows the temporal course of the gas pulses, by means of which the medical gas NO or the gas mixture (NO/N₂), (NO/He) is fed into the respiratory gas feed line 24. In the exemplary embodiment shown, it can be seen that, during the inhalation phases of the patient, the gas flow through the valves 54 to 60 or through the valve bank 52 at constant pulse width is varied by a specific selection and/or combination of different valves 54 to 60.

FIG. 7 shows a representation of the temporal course of the respiration of the mechanically ventilated patient 14 and of the administration of the medical gas according to a fourth exemplary embodiment of the invention. The fourth exemplary embodiment differs from the exemplary embodiment shown in FIG. 6 in that gas pulses having a greater pulse width are administered during the inhalation phases than during the exhalation phases. Thus, the administered amount of medical gas is increased. More particularly, what can be achieved by this, even with high flow velocities of the respiratory gas, is that the amount of administered medical gas is proportional to the flow velocity.

In other exemplary embodiments, the amount of gas administered in one gas pulse can be further varied in that the individual pulse widths, with which the valves 54 to 60 for producing a gas pulse are controlled, are different, and so at least two valves 54 to 60 deliver gas pulses of different pulse width. As a result, a total gas pulse is produced which has been produced from two subpulses of different pulse width. The total gas pulse then has a stepped course, which is fed into the respiratory gas feed line 24. In a specific embodiment of the third and fourth exemplary embodiment, the pulse frequencies during the inhalation phases are twice as high as in the exhalation phase. For example, the pulse frequency can be 208 gas pulses per minute during the inhalation phase and 104 gas pulses per minute during the exhalation phase. Alternatively, the pulse frequency can be 104 gas pulses per minute during the inhalation phase and 52 gas pulses per minute during the exhalation phase. Depending on the rise in the amount of gas inhaled at the start of an act of inhalation by the patient 14, i.e., depending on the flow at the start of the act of inhalation and/or the temporal course of the respiratory gas flow, it is possible for the length of an inhalation of the patient 14 and/or the course of the inhalation of the patient 14 to be empirically determined and, in line with the estimated course for each gas pulse during an inhalation, for an amount of the medical gas to be fed into the respiratory gas feed line 24 by this gas pulse to be defined. The defined amount of gas is then fed into the respiratory gas feed line 24 by appropriate control of the solenoid valves 54 to 60.

In an alternative embodiment of the invention, a closed circuit system is formed, and so the gas mixture exhaled by the patient 14 remains in the closed circuit system. Thus, the medical gas not taken up by the patient also remains in the circuit system. Such closed circuits are used especially during anesthesia of the patient 14. During anesthesia, the patient 14 is connected to an anesthesia machine. The control unit 48 is connected to the anesthesia machine via an interface. The anesthesia machine comprises at least one sensor for determining the start of a breath of the patient 14 and a sensor for determining the volume of gas mixture inhaled in said breath. The anesthesia machine transmits, via the interface, data containing information concerning the start of the breath and the inhaled volume of gas mixture to the control unit 48, which, as a function of said data, determines the amount of the medical gas injecting via the valves 54 to 60 such that as much medical gas is injected for it to be completely or at least almost completely taken up by the patient 14 in the breath, and so no accumulation of the medical gas occurs in the gas mixture of the closed circuit system. The control unit 48 controls the solenoid valves 54 to 60 in particular such that the amount of medical gas to be injected is injected within a short time at the start of the breath. Thus, an accumulation of the medical gas in the gas mixture is avoided and, as a result, reactions with other substances in the closed circuit system are, for example, avoided.

FIG. 8 shows a diagram of a device 300 for administering a medical gas NO to a patient 14 mechanically ventilated by means of a ventilator 12 according to a fifth embodiment of the invention. FIG. 9 shows a diagram of a device 400 for administering at least one medical gas NO according to a sixth embodiment of the invention. In the fifth embodiment according to FIG. 10, the carrier gas used for the medical gas is N₂ (nitrogen), whereas in the sixth embodiment according to FIG. 9, the carrier gas used is He (helium).

In contrast to embodiments 1 to 3, in embodiments 5 and 6, the medical gas NO is supplied directly to the patient feed line 30 via the gas line and not to the respiratory gas line 24. What is achieved by this is that the medical gas is supplied to the patient 14 as near as possible, and so it can be taken up by the patient 14 without a large time delay directly after it has been supplied via the gas feed line 40. This is advantageous especially when treating infants, for whom it is very important that the medical gas NO is supplied at the defined times during the respiration of the infant.

Furthermore, embodiments 5 and 6 differ from the first three embodiments in that the measurement line 41 removes the gas mixture to be analyzed not from the patient feed line 30, but rather from the waste air line 28. Via the measurement line 41, the gas is supplied to the measurement and evaluation unit 44 of the metering device 20.

As shown in FIG. 10, in both the fifth and sixth embodiment of the invention, the medical gas NO is supplied in a burst via a single gas pulse 402, 404 at the start of each inhalation phase. Thus, the amount of medical gas NO to be taken up by the patient 14 is fed immediately at the start of each inhalation phase, and so it is immediately made available for the uptake by the patient 14. Thus, much medical gas NO can be taken up at the start of the inhalation phase, and so it can penetrate deep into the lungs and can thus be taken up effectively by the patient 14. Furthermore, this supplying of the total amount of medical gas NO in a burst at the start of each inhalation phase has the advantage that very little medical gas NO is supplied unused during an apnea phase and/or an exhalation phase, during which it cannot be taken up by the patient 14.

FIG. 10 shows a first inhalation phase between time 0 and time t1 and a second inhalation phase between time t3 and time t4. As can be seen from the lower graph, the medical gas is supplied at the start of the first inhalation phase, i.e., just after time 0, by the gas pulse indicated by the reference symbol 402. At the start of the second inhalation phase, just after the starting time t3 of the inhalation phase, the medical gas is supplied by the gas pulse 404.

In a preferred embodiment of the invention, the ventilator 12 determines a flow profile of the respiration of the patient 14, as shown in the upper graph in FIG. 10. From the flow profile, the time of a future or of a current inhalation phase is determined as a function of the times (0, t3) of at least two immediately preceding inhalation phases. The control unit 48 controls the solenoid valves 54 to 60 such that the amount of the medical gas NO to be fed is fed in a burst via a gas pulse 402, 404 at the start of the determined inhalation phase. Furthermore, the gas volume of at least one preceding inhalation phase, preferably multiple preceding inhalation phases, is determined. As a function of said determined gas volume(s), the gas volume of the future or current inhalation phase is determined and, as a function thereof, the amount of medical gas NO to be fed into the patient feed line 30 is defined. The amount of medical gas NO to be fed is in particular defined such that the greater it is, the greater the expected gas volume of the future or current inhalation phase.

The gas volume of the preceding inhalation phases is determined in particular from the flow profile. For this purpose, the area enclosed by the curve determined during the inhalation phase and by the t axis is determined in particular. The expected gas volume of the future or current inhalation phase is determined in particular by averaging a preset number of preceding inhalation phases. Alternatively, the expected gas volume of the future or current inhalation phase can also be determined by means of pattern recognitions of the preceding inhalation phases, by means of which periodically recurring fluctuations in the respiration of the patient 14 can be determined for example.

As can be seen from FIG. 10, the gas volume of the first inhalation phase (0 to t1) is greater than the gas volume of the second inhalation phase (t3 to t4). Accordingly, the control unit 48 controls the solenoid valves 54 to 60 such that the first gas pulse 402 is greater than the second gas pulse 404. The size of the gas pulses 402, 404, i.e., the amount of medical gas which is supplied to the patient feed line 30 by the respective gas pulse 402, 404, can be set by the pulse duration of the gas pulses 402, 404 and by the flow volume of the medical gas during the respective gas pulses 402, 404.

In a further alternative embodiment of the invention, the medical gas is taken up in a carrier gas, more particularly helium. This reduces time delays in the transport of the medical gas through the lines, and so a precise control of the inspiration times is possible. This is necessary especially in the treatment of infants, since, during their treatment, even delays of 100 ms in the inspiration times may be critical with respect to the success or failure of the therapy. The ventilator 12 comprises a sensor for calculating the gas volume of a breath of the patient 14 and a sensor for determining the temporal start of a breath. The ventilator 12 is connected to the metering device 20 via a data interface, wherein data containing information concerning the volume of the last breath of the patient 14 and data containing information concerning the times of at least the last two breaths of the patient 14 are transmitted via the interface. The control unit 48 determines in real-time, as a function of these data, the start of the next breath of the patient 14 and controls, as a function of the calculated start of the breath and of at least the gas volume of the last breath, the solenoid valves 54 to 60 such that the injecting amount of medical gas is injected in a burst at the start of the next breath. Injection in a burst is understood to mean in particular that the medical gas is injected within a very short time. For this purpose, the control unit 48 opens the solenoid valves 54 to 60 as far as possible at the start of the breath.

To achieve that the therapy gas is fed into without delay in case of very short inhalation times either nitrogen (N₂) or helium (He) is used as carrier gas for the active substance (NO).

Although the invention above has been described in connection with preferred embodiments of the invention, it will be evident for a person skilled in the art that several modifications are conceivable without departing from the invention as defined by the following claims.

What is claimed is:

1. A method for administering at least one medical gas to a patient mechanically ventilated by means of a ventilator,
in which a first end of a first line supplying at least respiratory gas from the ventilator, a first end of a second line discharging at least exhaled gas from the patient and a first end of a patient feed line are connected to one another via at least one connecting piece, and
in which the medical gas is introduced into the patient feed line,
wherein a gas source for providing the medical gas to be administered and the patient feed line are connected via at least two regulating means arranged in parallel, wherein a connection is established between the gas source and the patient feed line via each regulating means in the opened state,
in that multiple gas pulses of the medical gas are fed successively into the patient feed line by means of the regulating means,
in that the temporal start of at least one inhalation phase is determined, and
in that the regulating means are controlled by means of a control unit such that a gas pulse is fed into the patient feed line at the determined start of the inhalation phase.

2. The method as claimed in claim 1, wherein exactly one gas pulse is fed during the inhalation phase.

3. The method as claimed in claim 1, wherein a pulse frequency or the pulse frequency of the gas pulses is 26, 52, 104 or 208 pulses/minute.

4. The method as claimed in claim 1, wherein the regulating means are controlled by means of the control unit such that an amount of gas defined in relation to a gas pulse is fed into the patient feed line.

5. The method as claimed in claim 1, wherein the medical gas contains NO, preferably NO and N₂ or NO and He.

6. The method as claimed in claim 1, wherein there are more than two, preferably four, regulating means.

7. The method as claimed in claim 1, wherein the regulating means in the opened state allow volume flows which are different from one another to pass through from the gas source to the patient feed line.

8. The method as claimed in claim 1, wherein the regulating means each comprise a solenoid valve.

9. The method as claimed in claim 1, wherein at least one restricting orifice for limiting the volume flow flowing through the regulating means is arranged upstream and/or downstream of at least one regulating means.

10. The method as claimed in claim 1, wherein regulating means of the same type are used and in that the volume flow flowing through the regulating means in the opened state differs owing to the provision of different flow resistances.

11. The method as claimed in claim 1, wherein the regulating means are switched between a completely opened state and a completely closed state.

12. The method as claimed in claim 1, wherein gas is removed from the second line and in that at least the proportion of the medical gas in the removed gas is determined.

13. The method as claimed in claim 12, wherein the gas is removed from the second line via a measurement line and supplied to an analysis unit for the detection of at least the proportion of the medical gas.

14. The method as claimed in claim 12, wherein the determined proportion of the medical gas, as the actual value, is compared with a target value, and in that, in the event of a determined deviation of the actual value from the preset target value, the amount of the medical gas introduced into the patient feed line during the gas pulse is adapted as a function of the comparative result, preferably regulated to the target value.

15. The method as claimed in claim 12, wherein at least the proportion of a reaction product of the medical gas, preferably an oxidation product of the medical gas, is detected, wherein the medical gas is preferably NO and the oxidation product is NO₂.

16. The method as claimed in claim 12, wherein the ventilator determines information concerning a flow profile of the respiration of the mechanically ventilated patient and in that the control unit determines the start of the inhalation phase as a function of the determined flow profile.

17. The method as claimed in claim 16, wherein the temporal start of multiple inhalation phases is determined, and in that one gas pulse is fed at each start of an inhalation phase.

18. The method as claimed in claim 16, wherein at least one exhalation phase and/or at least one apnea phase are determined from the flow profile, and in that no gas pulse is fed into the patient feed line during the exhalation phase and/or the apnea phase.

19. The method as claimed in claim 16, wherein data containing information concerning the times of at least two preceding inhalation phases are determined, and in that the start of the inhalation phase is determined as a function of said data.

20. The method as claimed in claim 16, wherein the gas volume of at least one preceding inhalation phase, preferably of multiple preceding inhalation phases, is determined, and in that the control unit determines, as a function of the gas volume(s), the gas volume of the determined inhalation phase, and in that the control unit defines the amount of medical gas which is fed via the gas pulse at the start of the determined inhalation phase as a function of the determined gas volume of the determined inhalation phase.

21. The method as claimed in claim 20, wherein the ventilator determines information concerning a flow profile of the respiration of the mechanically ventilated patient and in that the control unit determines the gas volume(s) of the preceding inhalation phase(s) as a function of the determined flow profile.

22. The method as claimed in claim 20, wherein the gas pulse is fed in a burst at the start of the determined inhalation phase.

23. A device for administering at least one medical gas to a patient mechanically ventilated by means of ventilator,
having a first line supplying at least respiratory gas from the ventilator,
having a second line discharging at least exhaled gas from the patient,
having a patient feed line, wherein a first end of the first line, a first end of the second line and a first end of the patient feed line are connected to one another via at least one connecting piece,
having a ventilator which produces a respiratory gas flow, and
having supply means for supplying the medical gas to the patient line,
wherein there are at least two regulating means arranged in parallel which connect a gas source for providing the medical gas to be administered and the patient line, wherein each regulating means in the opened state establishes a connection between the gas source and the patient line, and
having a control unit which controls the regulating means such that at least one regulating means successively feeds multiple gas pulses of the medical gas into the patient line,
in that the control unit determines the temporal start of at least one inhalation phase, and
in that the control unit controls the regulating means such that they feed a gas pulse into the patient feed line at the determined start of the inhalation phase.

24. The device as claimed in claim 23, wherein as a function of the amount of the medical gas to be introduced into the patient feed line, the control unit selects a regulating means to be opened for producing the gas pulse or selects multiple regulating means to be opened, and the pulse duration defines, as a function of the pressure difference present between a feed line of the gas source and the patient line, gas flow to be expected in the case of the selected regulating means or in the case of the multiple selected regulating means.
